(19) United States
(12) Patent Application Publication
(10) Pub. No.: US 2024/0316315 A1
(43) Pub. Date: Sep. 26, 2024

(54) ELONGATED MEDICAL INSTRUMENTS WITH FLEXIBILITY-ENHANCING FEATURES

(71) Applicant: Transit Scientific, LLC, Salt Lake City, UT (US)

(72) Inventor: Shawn P. Fojtik, Park City, UT (US)

(21) Appl. No.: 18/670,630

(22) Filed: May 21, 2024

Related U.S. Application Data
(63) Continuation-in-part of application No. 16/261,557, filed on Jan. 29, 2019, now Pat. No. 11,986,605.
(60) Provisional application No. 62/623,180, filed on Jan. 29, 2018.

(51) Int. Cl. A61M 25/00

(52) U.S. Cl. CPC A61M 25/0051; A61M 25/0013

(57) ABSTRACT

An elongated medical device includes an elongated tubular element with a series of flexibility enhancing features along a length of the elongated tubular element. Each flexibility enhancing feature may include a series of cuts. The cuts of one flexibility enhancing feature may be offset relative to the cuts of a longitudinally adjacent flexibility enhancing feature. The flexibility enhancing features may be grouped into two or more interleaved sets, with each set of circumferential cuts being offset relative to the next set of circumferential cuts. Methods for manufacturing such elongated medical devices are also disclosed.

### CROSS-REFERENCE TO RELATED APPLICATION

This application is a continuation-in-part of U.S. application Ser. No. 16/261,557, filed Jan. 29, 2019, titled ELONGATED MEDICAL INSTRUMENTS WITH FLEXIBILITY-ENHANCING FEATURES (“the '557 Provisional Application”), which claims benefit of priority to the Jan. 29, 2018 filing date of U.S. Provisional Patent Application No. 62/623,180, titled ELONGATED MEDICAL INSTRUMENTS WITH FLEXIBILITY-ENHANCING FEATURES (“the '180 Provisional Application”). The entire disclosures of the '180 Provisional Application and the '557 Application are hereby incorporated herein.

### TECHNICAL FIELD

This disclosure relates generally to elongated medical devices with features that enhance flexibility at one or more locations along the lengths of such elongated medical devices and to arrangements of flexibility-enhancing features along the lengths of elongated medical instruments. Some embodiments according to this disclosure relate to flexibility-enhancing features for hypotubes and to hypotubes that include the flexibility-enhancing features.

### RELATED ART

Hypodermic tubes, or “hypotubes,” are useful for a variety of purposes. In the medical device industry, hypotubes may facilitate the introduction of catheters through a patient's anatomy. Hypotubes may be manufactured in a variety of lengths and with outer diameters ranging from about 0.120 inch (11 gauge) to 0.005 inch (36 gauge).

Medical hypotubes are commonly formed from stainless steel (e.g., 304 stainless steel, 316 stainless steel, etc.). Stainless steel hypotubes may enhance the ability of a catheter to glide (e.g., push, track, and torque) through a subject's anatomy, but are known for their tendency to kink, particularly when forced through tortuous paths. Nitinol has also been used to form hypotubes. While nitinol hypotubes are kink-resistant, they are much more expensive to manufacture (particularly in developmental stages and in small quantities) than stainless steel hypotubes.

### DISCLOSURE

An elongated medical device according to this disclosure includes an elongated tubular element with a series of flexibility enhancing features along a length of the elongated tubular element, which may be referred to as a “longitudinal series” of flexibility enhancing features.

The elongated tubular element may comprise any suitable type of elongated tubular element, including elongated tubular elements that could benefit from enhanced flexibility. In some embodiments, the elongated tubular element may comprise a hypotube. The hypotube may be formed from any suitable material, such as stainless steel (e.g., 304 stainless steel, 316 stainless steel, etc.). A stainless steel hypotube that includes flexibility-enhancing features, such as a series of adjacent cuts that extend around a portion of a circumference of the hyptotube, as disclosed herein, could be less likely to kink than a conventional stainless steel hypotube. A hypotube according to this disclosure could also be formed from any of a variety of other metal alloys, including, but not limited to, nickel-titanium alloys, such as so-called “nitinol” (nickel-titanium Naval Ordinance Laboratory) phase change alloys. In other embodiments, the elongated tubular element of an elongated medical device according to this disclosure could be formed from a polymer. Regardless of the material from which the elongated tubular element is formed, it includes a wall that defines an outer surface of the tubular element and a lumen that extends through at least a portion of a length of the tubular element.

The longitudinal series of flexibility enhancing features are configured and arranged in a manner that enhances a flexibility of the elongated tubular element. Each flexibility enhancing feature may comprise a series of cuts (i.e., a plurality, or two or more, cuts) arranged around a circumference of a portion of the elongated tubular element. The series of cuts may be arranged around a circumferential location around the elongated tubular element, normal to a longitudinal axis of the elongated tubular element; such a series of cuts may be referred to as a “circumferential series” of cuts and as “circumferentially aligned cuts.” Alternatively, the series of cuts may arranged helically around the elongated tubular element; such a series of cuts may be referred to as a “helical series” of cuts and as “helically aligned cuts.” In various embodiments, each flexibility enhancing feature may include two cuts, three cuts, four cuts, etc., in a single turn (circumferentially or helically) around the elongated tubular element. Collectively, the series of cuts that defines such a flexibility enhancing feature may extend around a majority of the distance (i.e., a turn) around the elongated tubular element (but not completely around the circumference, as such an arrangement would sever the elongated tubular element) while enabling the elongated tubular element to retain sufficient structural integrity to facilitate its intended use as an elongated medical instrument or as part of an elongated medical instrument.

The arc lengths of the cuts that define each series of cuts of such a flexibility enhancing feature may be the same as one another. The solid regions of the elongated tubular element that alternate with the cuts of a series of cuts of a flexibility enhancing feature, or the distance or arc length of each solid region between adjacent (end-to-end), aligned cuts, may also have the same lengths. As alternatives, the arc lengths of adjacent, aligned cuts of a particular flexibility enhancing feature may differ from one another and/or the arc lengths of the solid regions between the adjacent, aligned cuts may differ from one another.

The cuts of one flexibility enhancing feature may be offset relative to the cuts of a longitudinally adjacent flexibility enhancing feature. Such a rotational offset may be fixed and follow a defined pattern. The flexibility enhancing features may be grouped into two or more interleaved sets, with each set of cuts being offset relative to the next set of cuts. In some embodiments, every n^{th} (e.g., 2^{nd}, 3^{rd}, 4^{th}, 5^{th}, etc.) flexibility enhancing feature of a group (e.g., n₁, n₂, n₃, etc.) may be rotationally offset relative to its preceding flexibility enhancing feature of the group (e.g., n₀, n₁, n₂, etc.) by a first, fixed rotational offset, or a first rotational angle. Without limitation, a first set of flexibility enhancing features of a series of flexibility enhancing features may include every odd numbered flexibility enhancing feature of the series, while a second set of flexibility enhancing features of the series of adjacent flexibility enhancing features may include every even numbered flexibility enhancing feature of the series, which may also be rotationally offset from each other. In some embodiments, the first rotational offset of one set of flexibility enhancing features (e.g., every n^{th} flexibility enhancing feature; e.g., where n=2, the 1^{st}, 3^{rd}, 5^{th}, etc., flexibility enhancing features) may be the same as the first rotational offset of another set of flexibility enhancing features (e.g., every n^{th}+1 flexibility enhancing feature; e.g., where n=2, the 2^{nd}, 4^{th}, 6^{th}, etc., flexibility enhancing features). In other embodiments, the first rotational offset of one set of flexibility enhancing features (e.g., every n^{th} flexibility enhancing feature; e.g., where n=2, the 1^{st}, 3^{rd}, 5^{th}, etc., flexibility enhancing features) may be different from the first rotational offset of a different set of flexibility enhancing features (e.g., every n^{th}+1 flexibility enhancing feature; e.g., where n=2, the 2^{nd}, 4^{th}, 6^{th}, etc., flexibility enhancing features; etc.).

The cuts of each flexibility enhancing feature may be rotationally offset from the cuts of its immediately preceding flexibility enhancing feature and by a second rotational offset, or a second rotational angle. The second rotational offset may differ from the first rotational offset. Thus, each set of flexibility enhancing features interleaved in series along the length of the elongated tubular element may be rotationally offset relative to every other set of flexibility enhancing features by the second rotational offset.

Elongated medical devices with flexibility enhancing features that are offset quasi-randomly or randomly, or that appear to have random rotational offsets relative to one another, are also within the scope of this disclosure.

Methods for defining flexibility enhancing features in an elongated tubular element are also disclosed. Such a method may employ laser cutting techniques, in which an elongated tubular element is introduced into a laser cutting device and moved longitudinally through the laser cutting device. More specifically, a drive element, or a so-called “chuck,” may engage the elongated tubular element along a portion of its length and may move the elongated tubular element through the laser cutting device. Movement of the elongated tubular element through the laser cutting device may be incremental (e.g., when forming cuts oriented perpendicular, or normal, to a longitudinal axis of the elongated tubular element, etc.) or continuous (e.g., when forming cuts that are oriented helically around the elongated tubular element, etc.). In embodiments where the elongated tubular element is advanced incrementally through the laser cutting device, a series of cuts may be made by a laser of the laser cutting device at each increment, as the elongated element is rotated about its longitudinal axis. In embodiments where the elongated tubular element is advanced continuously through the laser cutting device, the elongated tubular element may also be continuously rotated about its longitudinal axis as the laser makes a series of cuts in the elongated tubular element.

In some embodiments, as the drive element of the laser cutting device reaches a final position along the distance it can travel through the laser cutting device, or its “length of travel” or “throw length,” the arc length of the final cut that is made before the drive element disengages the elongated tubular element, returns to its initial position along its length of travel, and re-engages the elongated tubular element may be reduced relative to the lengths of the arcs of the other cuts in the same series and of other flexibility enhancing features that are defined while the drive element travels from its initial position to its final position within the laser cutting device. The reduction in length of that cut may result in a corresponding decrease in the amount of time required to define a longitudinal series of flexibility enhancing features in the elongated tubular element. In some embodiments, this technique has been shown to reduce laser cutting time by as much as 40% with no measurable impact on the flexibility of the resulting elongated medical device.

Other aspects of the disclosed subject matter, as well as features and advantages of various aspects of the disclosed subject matter, should become apparent to one of ordinary skill in the art through consideration of the ensuing description, the accompanying drawings, and the appended claims.

### BRIEF DESCRIPTION OF THE DRAWINGS

In the drawings:

FIG. 1 is a perspective view of a portion of an embodiment of an elongated medical device according to this disclosure;

FIG. 2 is an engineering drawing of a portion of an embodiment of an elongated medical device according to this disclosure;

FIG. 3 illustrates the flexibility of an embodiment of an elongated medical device according to this disclosure; and

FIG. 4 provides a representation of a method of manufacturing an elongated medical device according to this disclosure.

### DETAILED DESCRIPTION

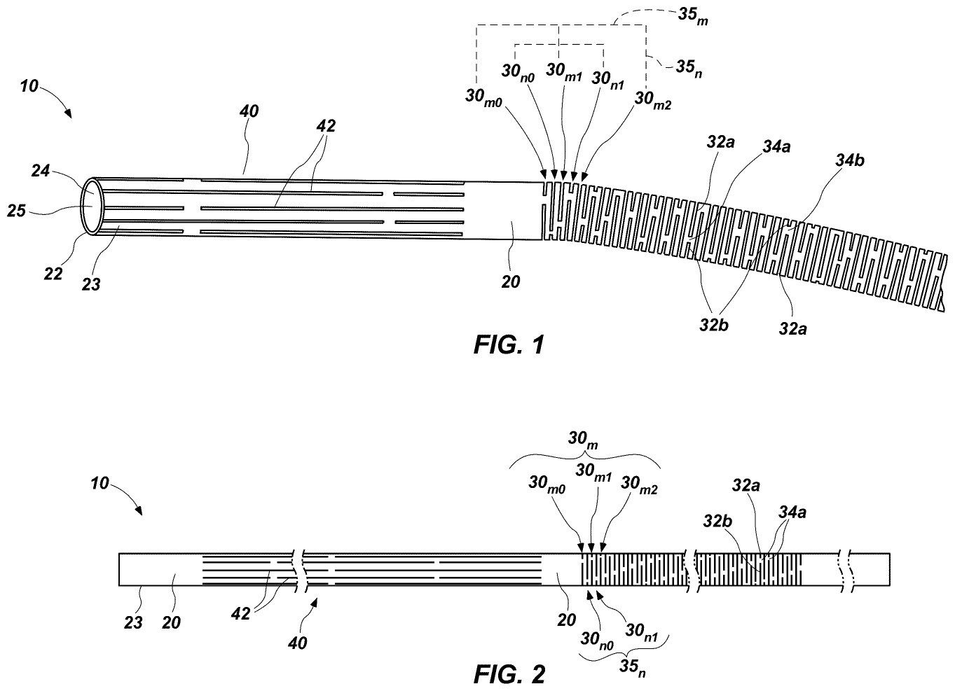

With reference to FIGS. 1 and 2, embodiments of an elongated medical device according to this disclosure are depicted, with FIG. 1 providing a partial perspective view of a portion of the elongated medical device 10 in a curved configuration or a bent configuration and FIG. 2 providing an engineering drawing of a portion of the elongated medical device 10 in a straight configuration. As illustrated, the elongated medical device 10 includes an elongated tubular element 20, a plurality of flexibility enhancing features 30 arranged along a length of the elongated tubular element 20, as well as other optional features.

The elongated tubular element 20 is defined by a wall 22, which is elongated and tubular in shape. The wall 22 includes an outer surface 23 and an inner surface 24 opposite from the outer surface 23. The outer surface 23 of the wall 22 defines an exterior of the elongated tubular element 20, as well as an exterior of the elongated medical device 10 of which the elongated tubular element 20 is a part. The inner surface 24 of the wall 22 defines a lumen 25 that extends through at least a portion of a length of the elongated tubular element 20 and, optionally, of the elongated medical device 10 of which the elongated tubular element 20 is a part. In some embodiments, the lumen 25 may extend through an entire length of the elongated tubular element 20 and, optionally, of the elongated medical device 10 of which the elongated tubular element 20 is a part.

A thickness of the wall 22 of the elongated tubular element 20 may, along with a material from which the elongated tubular element 20 is formed and other dimensions of the elongated tubular element 20 (e.g., its outer diameter (OD), its inner diameter (ID), etc.), impart the elongated tubular element 20 with sufficient structural integrity to enable the lumen 25 of the elongated tubular element 20 to remain open and even substantially retain its cross-sectional (taken normal to the longitudinal axis through the center of the lumen 25) shape (e.g., circularity, etc.) during use of the elongated medical device 10. Additionally, the thickness of the wall 22 may prevent kinking of the elongated tubular element 20 and collapsing of the lumen 25 during use of the elongated medical device 10 (e.g., as the elongated medical device 10 flexes, or curves, along its length, etc.). Thus, the wall 22 may have a thickness that prevents any disruption in the flow of fluids (e.g., liquids, gases, etc.) through the lumen 25 of the elongated tubular element 20 during use of an elongated medical device 10 of which the elongated tubular element 20 is a part. In various embodiments, the wall 22 of the elongated tubular element 20 may have a thickness (i.e., a distance along a radius of the elongated tubular element 20) of about 0.0015 inch (about 38 μm) to about 0.006 inch (about 150 μm).

In addition to the thickness of its wall 22, the OD and ID of the elongated tubular element 20 may have any suitable measurements. As examples, the ID of the elongated tubular element 20, or the diameter of the lumen 25 of the elongated tubular element 20, may be about 0.002 inch (about 50 μm) to about 0.070 inch (about 1.8 mm); the OD of the elongated tubular element 20 may be about 0.005 inch (about 125 μm) to about 0.080 inch (about 2 mm).

The elongated tubular element 20 may, in some embodiments, comprise a hypotube, which may be formed from a suitable metal or metal alloy. Examples of such materials include, but are not limited to, stainless steel (e.g., 304 stainless steel, 316 stainless steel, etc.) and nickel-titanium alloys, including nickel-titanium alloys whose flexibility varies depending upon their temperature (e.g., flexible at ambient temperature, rigid at elevated temperature (e.g., body temperature, etc.); etc.). Alternatively, the elongated tubular element 20 may be formed from a polymer.

With continued reference to FIGS. 1 and 2, each flexibility enhancing feature 30 of an elongated medical device 10 according to this disclosure may comprise cuts 32a, 32b, etc., which may also be individually referred to herein as a “cut 32” and collectively referred to herein as “cuts 32.” Each cut 32 may extend from the outer surface 23 of the wall 21 of the elongated tubular element 20 toward the inner surface 24 of the wall 22 of the elongated tubular element 20. The cuts 32 may extend partially through a thickness of the wall 22 or completely through the thickness of the wall 22.

Each flexibility enhancing feature 30 may comprise a series of cuts 32a, 32b, etc., (i.e., a plurality, or two or more, cuts 32). In various embodiments, each flexibility enhancing feature 30 may include a series of two cuts 32, three cuts 32, four cuts 32, etc. Collectively, the series of cuts 32a, 32b, etc., that defines such a flexibility enhancing feature 30 may extend a majority of the distance around, or a majority of a turn around, the elongated tubular element 20, with gaps, or solid regions 34a, 34b, etc., of the elongated tubular element 40, being located between adjacent cuts 32a and 32b, etc., of an end-to-end series of cuts 32. Each solid region 34a, 34b, etc., may also be individually referred to herein as a “solid region 34,” and solid regions 34a, 34b, etc., may be collectively referred to herein as “solid regions 34.” In some embodiments, the cuts 32a, 32b, etc., that define a flexibility enhancing feature 30 may collectively extend around about 75% or more of the distance around the elongated tubular element 20, with solid regions 34a, 34b, etc., of the elongated tubular element 20 defining about 25% or less of the distance around the elongated tubular element 20. As another option, the series of cuts 32a, 32b, etc., of a flexibility enhancing feature 30 may extend around about 80% or more of the distance around the elongated tubular element 20, with solid regions 34a, 34b, etc., of the elongated tubular element 20 defining about 20% or less of the distance around the elongated tubular element 20. Alternatively, the series of cuts 32a, 32b, etc., of a flexibility enhancing feature 30 may extend around about 90% or more of the distance around the elongated tubular element 20, with solid regions 34a, 34b, etc., of the elongated tubular element 20 defining about 10% or less of the distance around the elongated tubular element 20. In other embodiments, the series of cuts 32a, 32b, etc., of a flexibility enhancing feature 30 may extend around about 95% or more of the distance around the elongated tubular element 20, with solid regions 34a, 34b, etc., of the elongated tubular element 20 defining about 5% or less of the distance around the elongated tubular element 20. In still other embodiments, the series of cuts 32a, 32b, etc., of a flexibility enhancing feature 30 may extend around about 98% or more of the distance around the elongated tubular element 20, with solid regions 34a, 34b, etc., of the elongated tubular element 20 defining about 2% or less of the distance around the elongated tubular element 20. As another option, the series of cuts 32a, 32b, etc., of a flexibility enhancing feature 30 may extend around about 99% or more of the distance around the elongated tubular element 20, with solid regions of the elongated tubular element 20 defining about 1% or less of the distance around the elongated tubular element 20. Ranges of cut regions and solid regions between any of the foregoing values are also within the scope of this disclosure.

The arc lengths of the cuts 32a, 32b, etc., that define each series of cuts of a flexibility enhancing feature 30 may be the same as one another. As an example, each flexibility enhancing feature 30 may include a pair of aligned cuts 32a and 32b. The aligned cuts 32a and 32b of each pair may define arcs around opposite sides of the elongated tubular element 20. The arc defined by each cut 32a, 32b of a pair is less than 180° around the elongated tubular element 20 (i.e., the pair of aligned cuts 32a and 32b does not extend completely around the elongated tubular element 20). The arc of each cut 32a, 32b of such a pair may extend at least 90°, at least 112.5°, at least 135°, at least 157.5°, at least 165°, about 170°, or at least 172.5° around the elongated tubular element 20.

The solid regions 34a, 34b, etc., of the elongated tubular element 20 that alternate with the cuts 32a, 32b, etc., of a series of cuts 32 of a flexibility enhancing feature 30, or the distance or arc length of each solid region 34a, 34b, etc., between aligned cuts 32a, 32b, etc., may also have the same arcs or lengths as one another. In the embodiments described in the preceding paragraph, the solid regions 34a and 34b between aligned cuts 32a and 32b may also define opposed arcs around the elongated tubular element 20. Respectively, the arc of each solid region 34a, 34b between aligned cuts 32a and 32b that extend at least 90° around the elongated tubular element 20 may be less than 90°; the arc of each solid region 34a, 34b between aligned cuts 32a and 32b that extend at least 112.5° around the elongated tubular element 20 may be less than 67.5°; the arc of each solid region 34a, 34b between aligned cuts 32a and 32b that extend at least 135° around the elongated tubular element 20 may be less than 45°; the arc of each solid region 34a, 34b between aligned cuts 32a and 32b that extend at least 157.5° around the elongated tubular element 20 may be less than 22.5°; the arc of each solid region 34a, 34b between aligned cuts 32a and 32b that extend at least 165° around the elongated tubular element 20 may be less than 15°; the arc of each solid region 34a, 34b between aligned cuts 32a and 32b that extend about 170° around the elongated tubular element 20 may be about 10°; and the arc of each solid region 34a, 34b between aligned cuts 32a and 32b that extend at least 172.5° around the elongated tubular element 20 may be less than 7.5°.

As alternatives, the arcs and arc lengths of adjacent, aligned cuts 32a, 32b, etc., of a particular flexibility enhancing feature 30 may differ from one another and/or the arc lengths of the solid regions 34a, 34b, etc., between the adjacent, aligned cuts 32a, 32b, etc., may differ from one another.

The cuts 32 of one flexibility enhancing feature 30 may be offset relative to the cuts 32 of a longitudinally adjacent flexibility enhancing feature 30. Such a rotational offset may be fixed and follow a defined pattern along a longitudinal series of the flexibility enhancing features of an elongated tubular element 20. In some embodiments, adjacent flexibility enhancing features 30 of a series of flexibility enhancing features 30 may be grouped into two or more interleaved sets, with each set 35ₘ of flexibility enhancing features 30 being offset relative to the next set 35ₙ of flexibility enhancing features 30. Without limitation, as depicted by FIGS. 1 and 2, a first set 35ₘ of flexibility enhancing features 30 of a series of adjacent flexibility enhancing features 30 may include every odd numbered flexibility enhancing feature 30 of the series, while a second set 35ₙ of flexibility enhancing features 30 of the series of adjacent flexibility enhancing features 30 may include every even numbered flexibility enhancing feature 30 of the series. Stated another way, the cuts 32 of every n^{th} (e.g., 2^{nd}, 3^{rd}, 4^{th}, 5^{th}, etc.) flexibility enhancing feature 30ₙ₁, 30ₙ₂, etc., of a set 35ₙ may be rotationally offset relative to the preceding flexibility enhancing feature 30ₙ₀, 30ₙ₁, etc., of set 35ₙ by a first, fixed rotational offset.

The amount of the first rotational offset, or the first rotational angle, between adjacent flexibility enhancing features 30 in the series of flexibility enhancing features 30 or sequential flexibility enhancing features 30 in a set 35 of flexibility enhancing features 30 that is interleaved with one or more other sets 35 of flexibility enhancing features 30 of the series may be about 90° or less (i.e., a quarter of a turn, or rotation, around the elongated tubular element or less). Series of flexibility enhancing features 30 in which two adjacent or sequential flexibility enhancing features 30 have smaller rotational offsets of 60° or less, 45° or less, or even 30° or less are also within the scope of this disclosure.

In some embodiments, such as that depicted by FIGS. 1 and 2, a series of adjacent cuts may have two or more sets of rotational offsets. Stated another way, the cuts 32 of each flexibility enhancing feature 30 (e.g., flexibility enhancing feature 30ₙ₀, etc.) may be rotationally offset from the cuts 32 of its immediately (longitudinally) preceding flexibility enhancing feature 30 (e.g., flexibility enhancing feature 30ₘ₀, etc.) by a second rotational offset, or a second rotational angle, that differs from the first rotational offset; thus, each set 35 (e.g., set 35ₙ, etc.) of flexibility enhancing features 30 in series along the length of the elongated tubular element 20 may be rotationally offset relative to every other set 35 (e.g., set 35ₘ, etc.) of flexibility enhancing features 30 by the second rotational offset. In some embodiments, the first rotational offset of one set 35 of flexibility enhancing features 30 (i.e., every n^{th} flexibility enhancing feature 30; e.g., where n=3, the 1^{st}, 4^{th}, 7^{th}, etc., flexibility enhancing features 30) may be the same as the first rotational offset of a different set 35 of flexibility enhancing features 30 (i.e., every n^{th}+1 flexibility enhancing feature 30; e.g., where n=3, the 2^{nd}, 5^{th}, 8^{th}, etc., flexibility enhancing features 30; every n^{th}+2 flexibility enhancing feature 30; e.g., where n=3, the 3^{rd}, 6^{th}, 9^{th}, etc., flexibility enhancing features 30; etc.). In other embodiments, the first rotational offset of one set 35 of flexibility enhancing features 30 (i.e., every n^{th} flexibility enhancing feature 30; e.g., where n=3, the 1^{st}, 4^{th}, 7^{th}, etc., flexibility enhancing features 30) may be different from the first rotational offset of a different set 35 of flexibility enhancing features 30 (i.e., every n^{th}+1 flexibility enhancing feature 30; e.g., where n=3, the 2^{nd}, 5^{th}, 8^{th}, etc., flexibility enhancing features 30; every n^{th}+2 flexibility enhancing feature 30; e.g., where n=3, the 3^{rd}, 6^{th}, 9^{th}, etc., flexibility enhancing features 30; etc.). For example, the sets 35 may be rotationally offset by a rotation of greater than 0° and less than 180° (e.g., by about 90°, by about 120°, by about 150°, etc.).

Each pair of adjacent flexibility enhancing features 30 in a series of flexibility enhancing features 30 may be longitudinally spaced within 0.030 inch of each other, within 0.020 inch of each other, within 0.010 inch of each other, or even within 0.005 inch of each other. The spacing between adjacent flexibility enhancing features 30 may be constant, it may be varied in a pattern, or it may appear to be random (e.g., a different distance between each adjacent pair of flexibility enhancing feature 30, etc.). The longitudinal distance between adjacent flexibility enhancing features 30, or the pitch, of a series of flexibility enhancing features 30 may define the flexibility of a portion of the elongated medical device 10 along with the series of flexibility enhancing features 30 is located. As the width of each cut 32 may determine how closely adjacent flexibility enhancing features 30 may be positioned adjacent to one another (e.g., flexibility enhancing features 30 with thinner cuts 32 may be positioned at a tighter, or higher, pitch, than flexibility enhancing features 30 with wider cuts 32, etc.), the widths of the cuts 32 and, thus, of the flexibility enhancing features 30 they define may also affect the flexibility of the elongated medical device 10.

In some embodiments, the series of adjacent flexibility enhancing features 30 may extend along a length of a substantial portion of the elongated tubular element 20. In other embodiments, one or more series of flexibility enhancing features 30 may be formed in relative small sections along the length of the elongated tubular element 30.

Cuts through the wall of elongated tubular element that enhance the flexibility of the elongated tubular element or a portion thereof may be used in combination with longitudinal cuts 42 that define a feature 40 at or near a distal end of the elongated tubular element 20. Various embodiments of features that may be defined by longitudinally extending cuts 42 through the wall of the elongated tubular element 20 are disclosed by U.S. patent application Ser. No. 16/174,205, filed on Oct. 29, 2018 and titled EXOSKELETON DEVICE WITH EXPANDABLE SECTION FOR SCORING (“the '205 Application”). The entire disclosure of the '205 Application is hereby incorporated herein.

In a specific embodiment, an elongated medical device 10 according to this disclosure includes an elongated tubular element 20 that comprises a hypotube with a series of flexibility enhancing features 30 that are spaced about 0.010 inch apart from one another, with each cut 32a, 32b of a pair of aligned cuts 32 extending about 165° to about 172.5° around the hypotube. Every odd numbered (e.g., third, fifth, seventh, etc.) flexibility enhancing feature 30 (e.g., flexibility enhancing feature 30ₘ₁, 30ₘ₂, etc.) is rotationally offset by about 30° from the previous odd numbered (e.g., first, third, fifth, etc.) flexibility enhancing feature 30 (e.g., flexibility enhancing feature 30ₘ₀, 30ₘ₁, etc.). Every even numbered (e.g., fourth, sixth, eighth, etc.) flexibility enhancing feature 30 (e.g., flexibility enhancing feature 30ₙ₁, etc.) is rotationally offset by about 30° from the previous even numbered (e.g., second, fourth, sixth, etc.) flexibility enhancing feature 30 (e.g., flexibility enhancing feature 30ₙ₀, etc.). Thus, the set 35ₘ of odd numbered flexibility enhancing features 30 is rotationally offset from the set 35ₙ of even numbered flexibility enhancing features 30 (e.g., by about 90°). More specifically, the second flexibility enhancing feature 30ₙ₀ may be offset from the first flexibility enhancing feature 30ₘ₁ (at 0°) by about 90° (at about 90°), the third flexibility enhancing feature 30ₘ₁ may be offset from the second flexibility enhancing feature 30ₙ₁ by about −60° (at about 30°), the fourth flexibility enhancing feature 30ₙ₂ may be offset from the third flexibility enhancing features 30ₘ₂ by about 90° (at about 120°), the fifth flexibility enhancing feature 30ₘ₃ may be offset from the fourth flexibility enhancing feature 30ₙ₂ by about −60° (at about 60°), etc.

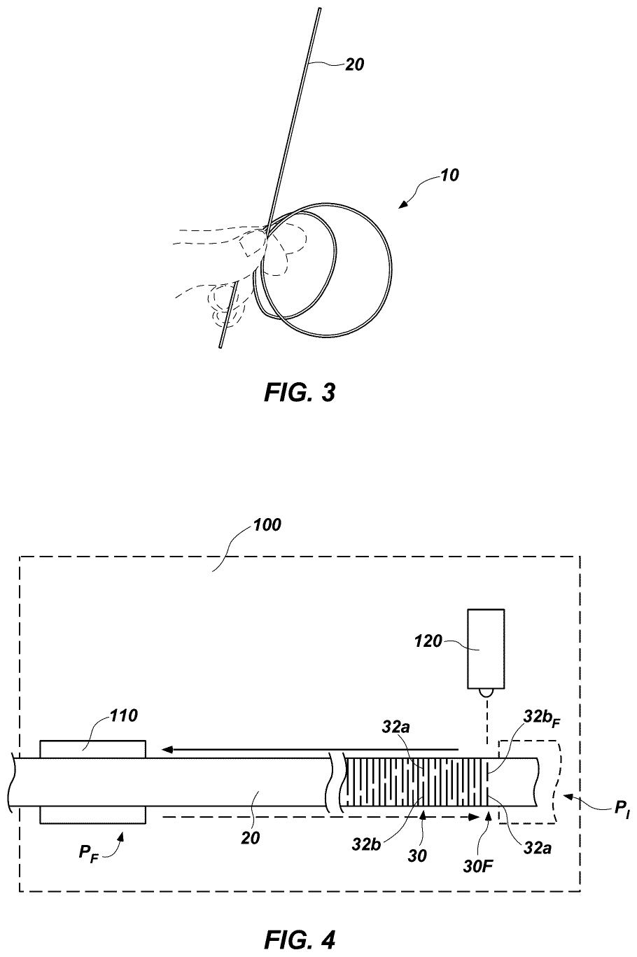

The flexibility enhancing features 30 of an elongated medical device 10 with an elongated tubular element 20 that comprises a stainless steel hypotube could render the elongated medical device 10 less likely to kink than a similar elongated medical device with conventionally arranged flexibility enhancing features. FIG. 3 illustrates the flexibility of such an embodiment of an elongated medical device 10, which includes flexibility enhancing features 30 (FIGS. 1 and 2) that are configured and arranged as disclosed herein.

Such an elongated medical device 10 may be used as an exoskeleton device around a catheter to enhance a usefulness of the catheter. For example, an elongated medical device 10 may impart a catheter with added rigidity, include features at or near its distal end that enable a balloon catheter (e.g., an angioplasty balloon, etc.) to score inner surfaces of a subject's vasculature or internal organs (e.g., the devices disclosed by the '205 Application), or may include features at or near its distal end that enable a balloon catheter to control delivery (e.g., infusion, etc.) of drugs or other substances to a particular location within a subject's body (e.g., his or her vasculature, internal organs, etc.).

Referring now to FIG. 4, an embodiment of a method for defining flexibility enhancing features 30 in an elongated tubular element 20 is disclosed. Such a method may employ laser cutting techniques, in which an elongated tubular element 20 is introduced into a laser cutting device 100 and moved longitudinally through the laser cutting device 100. More specifically, a drive element 110, or a so-called “chuck,” of the laser cutting device 100 may engage the elongated tubular element 20 along a portion of its length and may move the elongated tubular element 20 through the laser cutting device 100. During each course of travel through the laser cutting device 100, the drive element 110 may move from an initial position P_{I} to a final position P_{F}. Movement of the elongated tubular element 20 through the laser cutting device 100 may be incremental or continuous. When the elongated tubular element 20 advances incrementally through the laser cutting device 100, the length of each increment may correspond to a distance adjacent flexibility enhancing features 30 are to be spaced apart from one another, or the pitch of the adjacent flexibility enhancing features 30. When the elongated tubular element 20 advances continuously through the laser cutting device 100, the pitch of the cuts may be defined by the rate at which the elongated tubular element 20 advances and the rate of rotation of the elongated tubular element 20 relative to a laser 120 of the laser cutting device 100 (e.g., the rate at which the elongated tubular element 20 rotates, the rate at which the laser 120 is rotated around the elongated tubular element 20, etc.) as the elongated tubular element 20 is advanced through the laser cutting device 100.

The laser 120 of the laser cutting device 100 may generate a laser beam capable of defining a series of cuts 32a, 32b, etc., that defines each flexibility enhancing feature 30 along the length of the elongated tubular element 20. Each series of aligned cuts 32a, 32b, etc., may be defined as the drive element 110 carries the elongated tubular element 20 through the laser cutting device 100, with each series of cuts 32a, 32b, etc., being made as the elongated tubular element 20 is rotated within the laser cutting device 100 and/or as the laser 120 moves around the elongated tubular element 20. When the drive element 110 carries the elongated tubular element 20 incrementally through the laser cutting device 100, each time the drive element 110 and, thus, the elongated tubular element 20 stop, a location, or a particular circumference, along the length of the elongated tubular element 20 that is in line with the laser 120 may be cut to define a series of aligned cuts 32a, 32b, etc. Alternatively, when the drive element 110 carries the elongated tubular element 20 continuously through the laser cutting device 100 and the elongated tubular element 20, each series of aligned cuts 32a, 32b, etc., may be defined while the drive element 110 carries the elongated tubular element 20 past the location of the laser 120 and at least one of the elongated tubular element 20 and laser 120 rotates once relative to the other. The width of each cut 32a, 32b, etc., may correspond to the width of the laser beam generated by the laser 120, which width may be adjusted.

As the drive element 110 of the laser cutting device 100 reaches the final position P_{F} of its travel through the laser cutting device 100, the arc length of the final cut 32b_{F} that is made before the drive element 110 disengages the elongated tubular element 20, returns to its initial position P_{I} along its length of travel, and re-engages the elongated tubular element 20 may be reduced relative to the lengths of the arcs of the other cuts 32a_{F} in the same series. As a result, only the final flexibility enhancing feature 30_{F} that is formed with each course of travel of the drive element 110 through the laser cutting device 100 (e.g., one out of hundreds or even thousands of flexibility enhancing features 30 that are defined as the drive element 110 travels along a path that is a few inches long (e.g., 6 inches, 12 inches, etc.); etc.) will include a final cut 32b_{F} that is shorter than its circumferentially aligned cut(s) 32 and than the cuts 32 of the other flexibility enhancing features 30 that were defined during the same course of travel of the drive element 110. Thus, while the proportion in the reduction in length of that cut 32b_{F} may result in a corresponding decrease in the amount of time required to define a longitudinal series of flexibility enhancing features 30 in the elongated tubular element 20, this technique may have no measurable impact on the flexibility of the resulting elongated medical device 10 (FIGS. 1-3).

Although the preceding description and the accompanying drawings are limited to a few specific embodiments, the specific embodiments that have been described and illustrated should not be construed as limiting the scope of any of the appended claims. Features from different embodiments may be employed in combination. All additions to, deletions from, and modifications of the disclosed subject matter that fall within the scopes of the claims are to be embraced by the claims.

What is claimed:

1. An elongated medical device, comprising:
an elongated tubular element with a wall defining an outer surface and a lumen extending through a length of the elongated tubular element; and
a flexibility enhancement comprising a series of cuts formed through the wall along at least a portion of a length of the elongated tubular element; and
a final cut at an end of the series of cuts, the final cut having a length that is shorter than a length of every cut of the series of cuts.

2. The elongated medical device of claim 1, wherein each cut of the series of cuts is aligned with at least one other cut of the series of cuts.

3. The elongated medical device of claim 2, wherein each cut of the series of cuts is circumferentially aligned with at least one other cut of the series of cuts.

4. The elongated medical device of claim 2, wherein each cut of the series of cuts is helically aligned with at least one other cut of the series of cuts.

5. The elongated medical device of claim 1, wherein each cut of the series of cuts defines an arc of at least 90° around the elongated tubular element.

6. The elongate medical device of claim 5, wherein the arc of each cut of the series of cuts is less than 180° around the elongated tubular element.

7. The elongated medical device of claim 1, wherein the series of cuts includes:
a first set of cuts, each first cut of the first set of cuts being offset from a consecutive first cut of the first set of cuts by a first rotational angle; and
a second set of cuts interleaved with the first set of cuts, each second cut of the second set of cuts being offset from a consecutive second cut by the first rotational angle, the second set of cuts being offset from the first set of cuts by a second rotational angle.

8. The elongated medical device of claim 7, wherein the second rotational angle differs from the first rotational angle.

9. The elongated medical device of claim 1, wherein the elongated tubular element comprises a hypotube.

10. The elongated medical device of claim 9, wherein the hypotube comprises stainless steel.

11. The elongated medical device of claim 9, wherein the hypotube comprises nitinol.

12. The elongated medical device of claim 1, wherein the elongated tubular element comprises a polymer.

13. An elongated medical device, comprising:
an elongated tubular element with a wall defining an outer surface and a lumen extending through a length of the elongated tubular element; and
a flexibility enhancement comprising a series of cuts formed through the wall along at least a portion of a length of the elongated tubular element, each cut of the series of cuts defining an arc around the elongated tubular element; and
a final cut at an end of the series of cuts and having a length that is shorter than a length of the arc of every cut of the series of cuts.

14. The elongated medical device of claim 13, wherein the length of the final cut has no measurable impact on a flexibility of the flexibility-enhancement of the elongated medical device.

15. A method for manufacturing an elongated medical device, comprising:
engaging an elongated tubular element with a drive element of a laser cutting device at a first location along a length of the elongated tubular element;
advancing the elongated tubular element through the laser cutting device;
while rotating at least one of the elongated tubular element and a laser, defining a series of cuts in the elongated tubular element, each cut of the series of cuts having a predetermined arc length;
repeating the advancing and the defining a plurality of times to define at least one flexibility enhancing feature in the elongated tubular element;
while defining a final series of cuts of the at least one flexibility enhancing feature, defining a final cut having an arc length less than the predetermined arc length;
disengaging the elongated tubular element from the drive element;
re-engaging the elongated tubular element with the drive element at a second location along the length of the of the elongated tubular element; and
further advancing the elongated tubular element through the laser cutting device.

16. The method of claim 15, wherein:
advancing comprises incrementally advancing the elongated tubular element through the laser cutting device; and
defining the series of cuts occurs between incremental advancements of the elongated tubular element.

17. The method of claim 16, wherein an incremental advancement distance of incrementally advancing the elongated tubular element through the laser cutting device corresponds to a distance between longitudinally adjacent series of cuts along the length of the elongated tubular element.

18. The method of claim 15, wherein:
advancing comprises continuously advancing the elongated tubular element through the laser cutting device; and
defining the series of cuts occurs while continuously advancing the elongated tubular element.

19. The method of claim 18, wherein a rate of continuously advancing the elongated tubular element through the laser cutting device at least partially defines a distance between longitudinally adjacent series of cuts in the elongated tubular element.

20. The method of claim 15, wherein defining the final cut reduces an amount of time required to completely advance the elongated tubular element through the laser cutting device.
